(19) United States
(12) Patent Application Publication
(10) Pub. No.: US 2003/0045490 A1
(43) Pub. Date: Mar. 6, 2003

(54) Therapeutic antisense phosphodiesterase inhibitors

(75) Inventors: Roderic M.K. Dale, Wilsonville, OR (US); Amy Arrow, Bethel, ME (US); Terry Thompson, West Linn, OR (US)

Correspondence Address: MORGAN LEWIS & BOCKIUS LLP, 1111 PENNSYLVANIA AVENUE NW, WASHINGTON, DC, 20004, US

(73) Assignee: Oligos Etc. Inc.

(21) Appl. No.: 10/076,597

(22) Filed: Feb. 19, 2002

Related U.S. Application Data
(62) Division of application No. 09/364,626, filed on Jul. 29, 1999, now abandoned.
Application No. 09/364,626 is a continuation-in-part of application No. 09/223,586, filed on Dec. 30, 1998, now abandoned.

(51) Int. Cl. A61K 48/00; A61K 31/5377; A61K 31/522; C07H 21/04; C07D 473/18; C07D 413/14

(52) U.S. Cl. 514/44; 514/234.5; 514/263.31; 536/23.2; 544/81; 544/244; 544/268

(57) ABSTRACT

This patent describes the invention of a series of novel therapeutic oligonucleotides targeted at inhibiting expression of genes coding for Phosphodiesterase 4. They are useful as analytical tools in the study of individual PDE isoforms and in the therapeutic treatment of depression, thrombosis, cystic fibrosis, gastric lesions, pulmonary hypertension, glaucoma, multiple sclerosis, atopic dermatitis, asthma and other allergic disorders as well as other illnesses in which an increase of cyclic AMP or a decrease in phosphodiesterase levels is useful.

This application is a continuation-in-part of our earlier filed application Ser. No. 09/223,586, filed Dec. 30, 1998, to which we claim priority under 35 U.S.C. §120 and which is incorporated herein by reference in its entirety.

### BACKGROUND OF THE INVENTION

The enzyme phosphodiesterase (PDE), along with adenylate cyclase and guanylate cyclase, are enzymes responsible for maintaining the correct balance of cyclic AMP and cyclic GMP in cells. There are multiple distinct phosphodiesterases (PDE 1 through PDE 9), most of which exist as two or more isozymes or splice variants that can differ in their cellular distribution, specificity toward hydrolysis of cAMP or cGMP, selective inhibition by various compounds, and sensitivity to regulation by calcium, calmodulin, cAMP, and cGMP (J. A. Beavo in Cyclic Nucleotide Phosphodiesterases: Structure, Regulation and Drug Action. Multiple Phosphodiesterase Isozymes: Background, Nomenclature, and Implications. Eds. Beavo, J. and Houslay, M. D., John Wiley and Son, New York, 1990, pp. 3-15 and T. J. Torphy et al., “Novel Phosphodiesterases Inhibitors for the Therapy of Asthma”, Drug News & Prospective, 6(4) May 1993, pp. 203-214). The PDE4 family, which is specific for cAMP, is composed of at least 4 isozymes (a-d), and multiple splice variants (Houslay, M. D., et al. in Advances in Pharmacology 44, Eds. J. August et al., p.225, 1998). In total, there may be over 20 PDE4 isoforms expressed in a cell specific pattern regulated by a number of different promoters.

PDE4 is present in the brain and major inflammatory cells and has been found in abnormally elevated levels in a number of diseases including atopic dermatitis or eczema, asthma, and hay fever (ASTI Connections, Vol. 8 #1 (1996) p. 3 and J. of Allergy and Clinical Immunology 70:452-457,1982). Disease states for which selective PDE4 inhibitors have been sought include: asthma, atopic dermatitis, depression, reperfusion injury, septic shock, toxic shock, endotoxic shock, adult respiratory distress syndrome, autoimmune diabetes, diabetes insipidus, multi-infarct dementia, AIDS, cancer, Crohn's disease, multiple sclerosis, cerebral ischemia, psoriasis, allograft rejection, restenosis, ulcerative colitis, cachexia, cerebral malaria, allergic rhino-conjunctivitis, osteoarthritis, rheumatoid arthritis, chronic bronchitis, eosinophilic granuloma, and autoimmune encephalomyelitis (Houslay et al., 1998). In individuals suffering from atopic diseases, elevated PDE4 activity can be detected in their peripheral blood mononuclear leukocytes, T cells, mast cells, neutrophils and basophils. This increased PDE activity decreases cAMP levels and results in a breakdown of cAMP control in these cells, which in turn results in increased immune response in the blood and tissues of affected individuals.

PDE inhibitors influence multiple functional pathways, act on multiple immune and inflammatory pathways, and influence synthesis or release of numerous immune mediators (J. M. Hanifin and S. C. Chan, “Atopic Dermatitis-Therapeutic Implication for New Phosphodiesterase Inhibitors, Monocyte Dysregulation of T Cells” in AACI News, 7/2, 1995; J. M. Hanifin et al., “Type 4 Phosphodiesterase Inhibitors Have Clinical and In Vitro Anti-inflammatory Effects in Atopic Dermatitis,” J. of Invest. Derm., 1996, 107.51-56 and Cohen, V. L. in INC's 7th Annual Conference on Asthma and Allergy (Oct. 27-28, 1997)—Phosphodiesterase 4 Inhibitors: Second Generation and Beyond). Clinical use of inhibitors of PDE4 have shown them to be broad spectrum anti-inflammatory agents with impressive activity in models of asthma and other allergic disorders, including atopic dermatitis and hay fever. PDE4 inhibitors that have been used clinically include theophylline, rolipram, denbufylline, CDP 840 (a tri-aryl ethane) and CP80633 (a pyrimidone). PDE4 inhibitors have been shown to influence eosinophil responses, decrease basophil histamine release, decrease IgE, PGE2, and IL10 synthesis, and decrease anti-CD3 stimulated IL4 production.

All of the PDE4 inhibitors developed to date have been small molecule compounds which exhibit central nervous system and gastrointestinal effects. Although the first generation of PDE4 inhibitors have been effective in reducing PDE4 activity and alleviating a number of the inflammatory problems caused by overexpression of this enzyme, their effectiveness has been limited by mechanism related side effects and none of them can be used systemically because of nausea and vomiting (ASTI Connections, Vol. 8 #1 (1996) p. 3 and Hanifin reference and Cohen, V. L. in INC's 7th Annual Conference on Asthma and Allergy (Oct. 27-28, 1997)—Phosphodiesterase 4 Inhibitors: Second Generation and Beyond).

Oligonucleotide therapy, i.e., the use of oligonucleotides to modulate the expression of specific genes, offers an opportunity to selectively modify the expression of genes without the undesirable non-specific toxic effects of more traditional therapeutics. The use of antisense oligonucleotides has emerged as a powerful new approach for the treatment of many diseases. The preponderance of the work to date has focused on the use of antisense oligonucleotides as antiviral agents or as anticancer agents (Wickstrom, E., Ed., Prospects for Antisense Nucleic Acid Therapy of Cancer and AIDS, New York: Wiley-Liss, 1991; Crooke, S. T., and Lebleu, B., Eds., Antisense Research and Applications, Boca Raton: CRC Press, 1993, pp. 154-182; Baserga, R., and Denhardt, D. T., 1992, Antisense Strategies, New York: The New York Academy of Sciences, Vol. 660; Murray, J. A. H., Ed., Antisense RNA and DNA, New York: Wiley-Liss, 1993, Agrawal et al., Proc. Natl. Acad. Sci. USA 85:7079-7083 (1988), Zamecnik et al., Proc. Natl. Acad. Sci. USA 83:4143-4146 (1986)).

There is a need to develop a therapeutic agent for modulating PDE4 that is effective but does not possess the side effects of the current PDE4 inhibitors. There is thus a need for a bioavailable, non-toxic therapeutic agent that specifically modulates PDE4.

### SUMMARY OF THE INVENTION

The present invention provides end-blocked acid resistant nucleic acids, e.g., end-blocked 2′-O-alkyl and 2′-O-alkyl-n(O-alkyl) oligonucleotides, for use in modulating PDE4 activity, either in vivo or in vitro. These PDE4 oligonucleotides exhibit substantial stability at low pH, substantial resistance to nuclease degradation, and binding specificity both in vivo and in vitro.

The oligonucleotides may be either ribonucleotides and/or deoxyribonucleotides, and may be targeted to DNA sequences involved in the expression of PDE4 (e.g., coding sequences, promoter sequences, enhancer sequences, etc.) or to PDE4 mRNA. These low toxicity, highly specific, acid stable, end-blocked nucleic acids represent an improved nucleic acid structure for therapeutic treatments of PDE4-mediated diseases. The 3′ or 3′ and 5′ acid stable, nuclease resistant ends confer improved bioavailability by increasing nuclease resistance.

The invention also provides pharmaceutical compositions comprised of oligonucleotides of the invention. These pharmaceutical compositions may include any pharmaceutically acceptable carrier. The pharmaceutical composition may also include additives such as adjuvants, stabilizers, fillers and the like.

The invention also provides methods of treating a patient in need of such treatment with a therapeutically effective amount of an oligonucleotide targeted to PDE4. In a preferred embodiment, the present invention provides a series of antisense oligonucleotides targeted to mRNAs encoding different PDE4 isozymes. The therapeutic effectiveness of an oligonucleotide targeted against PDE4D is demonstrated herein using data from preliminary human trials.

The invention also comprises methods of reducing the activity of one or more PDE4 enzyme comprising treating a patient with one or more antisense oligonucleotides. Preferably the oligonucleotide is targeted to mRNA coding for PDE4.

It is an advantage of the oligonucleotides of the invention that the acid stable ends confer an improved stability on the modified nucleic acids in an acidic environment (e.g., the stomach, with a pH of 1 to 2), and thus increase bioavailability of the oligonucleotides in vivo.

It is another advantage of the nucleic acids of the invention that they bind with specificity to PDE4 target sequences in vivo and in vitro.

It is another advantage of the invention that the end-blocked nucleic acids are non-toxic to a subject treated with the modified nucleic acids. The modified nucleic acids of the present invention, e.g., 2′-O-alkyl and 2′-O-alkyl-n(O-alkyl) oligonucleotides, do not display side effects commonly caused by therapeutic administration of regular polyanionic oligonucleotides, such as increased binding to serum and other proteins, stimulation of serum transaminases, decreases in platelet counts, and the like.

It is yet another advantage that the acid stable ends confer an improved stability on the PDE4 oligonucleotides in the acid environments of lysosomal vesicles in macrophages and neutrophils.

It is a further advantage that the PDE4 oligonucleotides of the present invention are readily encapsulated in charged liposomes.

It is a further advantage that the PDE4 oligonucleotides have low toxicity, i.e., mice parenterally treated with a PDE4 oligonucleotide of the invention exhibit an LD₅₀ of less than one at 400 mg/ll.

These and other objects, advantages, and features of the invention will become apparent to those skilled in the art upon reading the details of the nucleic acids and uses thereof as more fully described below.

### DETAILED DESCRIPTION OF PREFERRED EMBODIMENTS

Before the present invention is described, it is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a construct” includes a plurality of such constructs and reference to “the oligonucleotide” includes reference to one or more oligonucleotides and equivalents thereof known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The terms “nucleic acid” and “nucleic acid molecule” as used interchangeably herein, refer to a molecule comprised of nucleotides, i.e., ribonucleotides, deoxyribonucleotides, or both. The term includes monomers and polymers of ribonucleotides and deoxyribonucleotides, with the ribonucleotide and/or deoxyribonucleotides being connected together, in the case of the polymers, via 5′ to 3′ linkages. However, linkages may include any of the linkages known in the nucleic acid synthesis art including, for example, nucleic acids comprising 5′ to 2′ linkages. The nucleotides used in the nucleic acid molecule may be naturally occurring or may be synthetically produced analogues that are capable of forming base-pair relationships with naturally occurring base pairs. Examples of non-naturally occurring bases that are capable of forming base-pairing relationships include, but are not limited to, aza and deaza pyrimidine analogues, aza and deaza purine analogues, and other heterocyclic base analogues, wherein one or more of the carbon and nitrogen atoms of the purine and pyrimidine rings have been substituted by heteroatoms, e.g., oxygen, sulfur, selenium, phosphorus, and the like.

The term “oligonucleotide” as used herein refers to a nucleic acid molecule comprising from about 1 to about 100 nucleotides, more preferably from 1 to 80 nucleotides, and even more preferably from about 4 to about 35 nucleotides.

The term “PDE4 oligonucleotide” as used herein refers to an oligonucleotide that is targeted to sequences that affect PDE4 expression or activity. These include, but are not limited to, PDE4 DNA coding sequences, PDE4 DNA promoter sequences, PDE4 DNA enhancer sequences, mRNA encoding PDE4, and the like.

The terms “modified oligonucleotide” and “modified nucleic acid molecule” as used herein refer to nucleic acids, including oligonucleotides, with one or more chemical modifications at the molecular level of the natural molecular structures of all or any of the nucleic acid bases, sugar moieties, internucleoside phosphate linkages, as well as molecules having added substituents, such as diamines, cholesteryl or other lipophilic groups, or a combination of modifications at these sites. The intemucleoside phosphate linkages can be phosphodiester, phosphotriester, phosphoramidate, siloxane, carbonate, carboxymethylester, acetamidate, carbamate, thioether, bridged phosphorarnidate, bridged methylene phosphonate, phosphorothioate, methylphosphonate, phosphorodithioate, bridged phosphorothioate and/or sulfone internucleotide linkages, or 3′-3′, 2′-5′ or 5′-5′ linkages, and combinations of such similar linkages (to produce mixed backbone modified oligonucleotides). The modifications can be internal (single or repeated) or at the end(s) of the oligonucleotide molecule and can include additions to the molecule of the intemucleoside phosphate linkages, such as cholesteryl, diamine compounds with varying numbers of carbon residues between amino groups and terminal ribose, deoxyribose and phosphate modifications which cleave or cross-link to the opposite chains or to associated enzymes or other proteins. Electrophilic groups such as ribose-dialdehyde could covalently link with an epsilon amino group of the lysyl-residue of such a protein. A nucleophilic group such as n-ethylmaleimide tethered to an oligomer could covalently attach to the 5′ end of an mRNA or to another electrophilic site. The term modified oligonucleotides also includes oligonucleotides comprising modifications to the sugar moieties such as 2′-substituted ribonucleotides, or deoxyribonucleotide monomers, any of which are connected together via 5′ to 3′ linkages. Modified oligonucleotides may also be comprised of PNA or morpholino modified backbones where target specificity of the sequence is maintained.

The term “nucleic acid backbone” as used herein refers to the structure of the chemical moiety linking nucleotides in a molecule. This may include structures formed from any and all means of chemically linking nucleotides. A modified backbone as used herein includes modifications to the chemical linkage between nucleotides, as well as other modifications that may be used to enhance stability and affinity, such as modifications to the sugar structure. For example an α-anomer of deoxyribose may be used, where the base is inverted with respect to the natural β-anomer. In a preferred embodiment, the 2′-OH of the sugar group may be altered to 2′-O-alkyl or 2′-O-alkyl-n(O-alkyl), which provides resistance to degradation without comprising affinity.

The term “acidification” and “protonation/acidification” as used interchangeably herein refers to the process by which protons (or positive hydrogen ions) are added to proton acceptor sites on a nucleic acid. The proton acceptor sites include the amine groups on the base structures of the nucleic acid and the phosphate of the phosphodiester linkages. As the pH is decreased, the number of these acceptor sites which are protonated increases, resulting in a more highly protonated/acidified nucleic acid.

The term “protonated/acidified nucleic acid” refers to a nucleic acid that, when dissolved in water at a concentration of approximately 16 A₂₆₀ per ml, has a pH lower than physiological pH, i.e., lower than approximately pH 7. Modified nucleic acids, nuclease-resistant nucleic acids, and antisense nucleic acids are meant to be encompassed by this definition. Generally, nucleic acids are protonated/acidified by adding protons to the reactive sites on a nucleic acid, although other modifications that will decrease the pH of the nucleic acid can also be used and are intended to be encompassed by this term.

The term “end-blocked” as used herein refers to a nucleic acid with a chemical modification at the molecular level that prevents the degradation of selected nucleotides, e.g., by nuclease action. This chemical modification is positioned such that it protects the integral portion of the nucleic acid, for example the coding region of an antisense oligonucleotide. An end block may be a 3′ end block or a 5′ end block. For example, a 3′ end block may be at the 3′-most position of the molecule, or it may be internal to the 3′ ends, provided it is 3′ of the integral sequences of the nucleic acid.

The term “substantially nuclease resistant” refers to nucleic acids that are resistant to nuclease degradation, as compared to naturally occurring or unmodified nucleic acids. Modified nucleic acids of the invention are at least 1.25 times more resistant to nuclease degradation than their unmodified counterpart, more preferably at least 2 times more resistant, even more preferably at least 5 times more resistant, and most preferably at least 10 times more resistant than their unmodified counterpart. Such substantially nuclease resistant nucleic acids include, but are not limited to, nucleic acids with modified backbones such as phosphorothioates, methylphosphonates, ethylphosphotriesters, 2′-0-methylphosphorothioates, 2′-O-methyl-p-ethoxy ribonucleotides, 2′-O-alkyls, 2′-O-alkyl-n(O-alkyl), 3′-O-alkyls, 3′-O-alkyl-n(O-alkyl), 2′-fluoros, 2′-deoxy-erythropentofuranosyls, 2′-O-methyl ribonucleosides, methyl carbamates, methyl carbonates, inverted bases (e.g., inverted T's), or chimeric versions of these backbones.

The term “substantially acid resistant” as used herein refers to nucleic acids that are resistant to acid degradation as compared to unmodified nucleic acids. Typically, the relative acid resistance of a nucleic acid will be measured by comparing the percent degradation of a resistant nucleic acid with the percent degradation of its unmodified counterpart (i.e., a corresponding nucleic acid with “normal” backbone, bases, and phosphodiester linkages). A nucleic acid that is acid resistant is preferably at least 1.5 times more resistant to acid degradation, at least 2 times more resistant, even more preferably at least 5 times more resistant, and most preferably at least 10 times more resistant than their unmodified counterpart.

The term “LD₅₀” as used herein is the dose of an active substance that will result in 50 percent lethality in all treated experimental animals. Although this usually refers to invasive administration, such as oral, parenteral, and the like, it may also apply to toxicity using less invasive methods of administration, such as topical applications of the active substance.

The term “alkyl” as used herein refers to a branched or unbranched saturated hydrocarbon chain containing 1-6 carbon atoms, such as methyl, ethyl, propyl, tert-butyl, n-hexyl and the like.

The term “sensitivity” as used herein refers to the relative strength of recognition of a nucleic acid by a binding partner, e.g., an oligonucleotide complementary to the nucleic acids of interest, i.e., PDE4. The recognition of the binding partner to the sequence of interest must be significantly greater than the recognition of background sequences, and preferably the strength of recognition of the binding partner is at least 10 times, more preferably at least 100 times greater, and even more preferably at least 500 times greater than recognition of background proteins.

The term “selectivity” as used herein refers to the preferential binding of a binding partner to a particular nucleic acid sequence. Preferably, a selective binding partner is at least 10 times, more preferably 100 times, and even more preferably 1000 times more likely to bind to its designated polynucleotide sequence than to any other background sequence.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes:

(a) preventing a disease from occurring in a subject which may be predisposed to the disease but has not yet been diagnosed as having it;

(b) inhibiting a disease, i.e., arresting its development; or

(c) relieving a disease, i.e., causing regression of the disease. The invention is generally directed toward treating patients by the administration of a nucleic acid sequence that will modulate expression of an endogenous gene in vivo.

By “therapeutically effective” amount is meant a nontoxic but sufficient amount of a compound to provide the desired therapeutic effect, in the present case, that dose of modified nucleic acid which will be effective in relieving, ameliorating, or preventing symptoms of the condition or disease being treated.

### GENERAL ASPECTS OF THE INVENTION

The concept of antisense has been well developed and a number of different antisense therapeutic drugs are in or have completed clinical trials. Antisense therapeutic compounds are oligonucleotides, preferably nuclease resistant, complementary to the mRNA coding for a particular protein. Antisense oligonucleotides generally interfere with the transcription or translation of the targeted gene and thereby reduce expression of the target gene. Because of the great specificity that is possible with antisense there are far fewer, if any, side affects.

In a preferred embodiment, the invention uses either neutral or protonated/acidified oligonucleotides that are substantially nuclease resistant. This embodiment includes oligonucleotides completely or partially derivatized by phosphorothioate linkages, 2′-deoxy-erythropentofuranosyl, 2′-fluoro, 2′-O-alkyl or 2′-O-alkyl-n(O-alkyl) phosphodiesters, p-ethoxy oligonucleotides, p-isopropyl oligonucleotides, phosporamidates, chimeric linkages, and any other backbone modifications. This embodiment also includes other modifications that render the oligonucleotides substantially resistant to endogenous nuclease activity. Additional methods of rendering an oligonucleotide nuclease resistant include, but are not limited to, covalently modifying the purine or pyrimidine bases that comprise the oligonucleotide. For example, bases may be methylated, hydroxymethylated, or otherwise substituted (e.g., glycosylated) such that the oligonucleotides comprising the modified bases are rendered substantially nuclease resistant.

In order to enhance the exonuclease resistance of the oligonucleotides of the invention, the 3′ and/or 5′ ends of the nucleic acid sequence are preferably attached to an exonuclease blocking function. For example, one or more phosphorothioate nucleotides can be placed at either end of the oligoribonucleotide. Additionally, one or more inverted bases can be placed on either end of the oligonucleotide, or one or more alkyl moieties, e.g., butanol-substituted nucleotides or chemical groups can be placed on one or more ends of the oligonucleotide. 5′ and 3′ end blocking groups may include one or more phosphorothioate nucleotides (but typically fewer than six), inverted base linkages, or alkyl, alkenyl, or alkynl groups or substituted nucleotides or 2′-O-alkyl-n(O-alkyl). A partial list of blocking groups includes inverted bases, dideoxynucleotides, methylphosphates, alkyl groups, aryl groups, cordycepin, cytosine arabanoside, 2′-methoxy, ethoxy nucleotides, phosphoramidates, a peptide linkage, dinitrophenyl group, 2′- or 3′-O-methyl bases with phosphorothioate linkages, 3′-O-methyl bases, fluorescein, cholesterol, biotin, acridine, rhodamine, psoralen, glyceryl, methyl phosphonates, a hexa-ethyloxy-glycol, butanol, hexanol, and 3′-O-alkyls. An enzyme-resistant butanol preferably has the structure OH-CH₂CH₂CH₂CH₂ (4-hydroxybutyl) which is also referred to as a C4 spacer.

Typically, the relative nuclease resistance of a nucleic acid can be measured by comparing the percent digestion of a resistant nucleic acid with the percent digestion of its unmodified counterpart (i.e., a corresponding nucleic acid with “normal” backbone, bases, and phosphodiester linkage). Percent degradation may be determined by using analytical HPLC to assess the loss of full length nucleic acids, or by any other suitable methods (e.g., by visualizing the products on a sequencing gel using staining, autoradiography, fluorescence, etc., or measuring a shift in optical density). Degradation is generally measured as a function of time.

Comparison between unmodified and modified nucleic acids can be made by ratioing the percentage of intact modified nucleic acid to the percentage of intact unmodified nucleic acid.

For example, if, after 15 minutes of exposure to a nuclease, 25% (i.e., 75% degraded) of an unmodified nucleic acid is intact, and 50% (i.e., 50% degraded) of a modified nucleic acid is intact, the modified nucleic acid is said to be 2 times (50% divided by 25%) more resistant to nuclease degradation than is the unmodified nucleic acid. Generally, a substantially nuclease resistant nucleic acid will be at least about 1.25 times more resistant to nuclease degradation than an unmodified nucleic acid with a corresponding sequence, typically at least about 1.5 times more resistant, preferably about 2 times more resistant, more preferably at least about 5 times more resistant, and even more preferably at least about 10 times more resistant after 15 minutes of nuclease exposure.

Percent acid degradation may be determined by using analytical HPLC to assess the loss of full length nucleic acids, or by any other suitable methods (e.g., by visualizing the products on a sequencing gel using staining, autoradiography, fluorescence, etc., or measuring a shift in optical density). Degradation is generally measured as a function of time.

Comparison between unmodified and modified nucleic acids can be made by ratioing the percentage of intact modified nucleic acid to the percentage of intact unmodified nucleic acid. For example, if, after 30 minutes of exposure to a low pH environment, 25% (i.e., 75% degraded) of an unmodified nucleic acid is intact, and 50% (i.e., 50% degraded) of a modified nucleic acid is intact, the modified nucleic acid is said to be 2 times (50% divided by 25%) more resistant to nuclease degradation than is the unmodified nucleic acid. Generally, substantially “acid resistant” nucleic acids will be at least about 1.25 times more resistant to acid degradation than an unmodified nucleic acid with a corresponding sequence, typically at least about 1.5 times more resistant, preferably about 1.75 more resistant, more preferably at least 5 times more resistant and even more preferably at least about 10 times more resistant after 30 minutes of exposure at 37° C. to a pH of about 1.5 to about 4.5.

Acidification of nucleic acids is the process by which protons (or hydrogen atoms) are added to the reactive sites on a nucleic acid. As the pH is decreased, the number of reactive sites protonated increases and the result is a more highly protonated/acidified nucleic acid. As the pH of the nucleic acid decreases, its bacterial inhibiting activity increases. Accordingly, the nucleic acids of the invention are protonated/acidified to give a pH when dissolved in water of less than pH 7 to about pH 1, or in preferred embodiments, pH 6 to about 1 or pH 5 to about 1. In other preferred embodiments, the dissolved nucleic acids have a pH from pH 4.5 to about 1 or, in a preferred embodiment, a pH of 4.0 to about 1, or, in a more preferred embodiment, a pH of 3.0 to about 1, or, in another preferred embodiment, a pH of 2.0 to about 1.

A first aspect of the present invention is a method of treating a patient requiring such treatment comprising administering one or more oligonucleotide(s) targeted to hybridize with one or more of the appropriate PDE4 mRNA(s) in a therapeutically effective amount. In a second aspect, the present invention is a method of treating a patient requiring such treatment comprising administering one or more oligonucleotide(s) targeted to hybridize with one or more of the appropriate PDE4 mRNA(s) in an amount effective to reduce expression of one or more of the PDE4 enzymes. Preferably, the antisense oligonucleotides are in a pharmaceutically acceptable carrier. The targeted PDE4 gene sequence or sequences are selected from the group consisting of the PDE4 genes, their isozymes and their splice variants.

The methods and compositions of the invention are useful as analytical tools in the study of individual PDE isoforms and in therapeutic treatment. The methods and compositions of the invention are useful for treating various diseases or disorders, including but not limited to: asthma, hay fever, atopic dermatitis, depression, reperfusion injury, septic shock, toxic shock, endotoxic shock, adult respiratory distress syndrome, autoimmune diabetes, diabetes insipidus, multi-infarct dementia, AIDS, cancer, Crohn's disease, multiple sclerosis, cerebral ischemia, psoriasis, allograft rejection, restenosis, ulcerative colitis, cachexia, cerebral malaria, allergic rhino-conjunctivitis, osteoarthritis, rheumatoid arthritis, chronic bronchitis, eosinophilic granuloma, and autoimmune encephalomyelitis. Particular embodiments of the present invention are directed towards the treatment of the above diseases.

Optionally, the presently described oligonucleotides may be formulated with a variety of physiological carrier molecules. The presently described oligonucleotides may also be complexed with molecules that enhance their ability to enter the target cells. Examples of such molecules include, but are not limited to, carbohydrates, polyamines, amino acids, peptides, lipids, and molecules vital to cell growth. For example, the oligonucleotides may be combined with a lipid, the resulting oligonucleotide/lipid emulsion, or liposomal suspension may, inter alia, effectively increase the in vivo half-life of the oligonucleotide. The use of cationic, anionic, and/or neutral lipid compositions or liposomes is generally described in International Publications Nos. WO 90/14074, WO 91/16024, WO 91/17424, U.S. Pat. No. 4,897,355, herein incorporated by reference.

Alternatively, oligonucleotides directed at PDE targets may also be protonated/acidified to function in a dual role as phosphodiesterase inhibitors and antibacterial agents. Accordingly, another embodiment of the presently described invention is the use of a PDE modulating therapeutic oligonucleotide that is additionally protonated/acidified to increase cellular uptake, improve encapsulation in liposomes, so it can also serve as an antibiotic.

Additionally, the oligonucleotide may be complexed with a variety of well established compounds or structures that, for instance, further enhance the in vivo stability of the oligonucleotide, or otherwise enhance its pharmacological properties (e.g., increase in vivo half-life, reduce toxicity, etc.).

Selection of Antisense Oligonucleotides

Antisense oligonucleotides may be targeted to particular functional domains of a gene or mRNA transcript. The potential targets include, but are not limited to, regulatory regions, the 5′-untranslated region, the translational start site, the translational termination site, the 3′-untranslated region, exon/intron boundaries and splice sites, as well as sequences internal to the coding region.

As a first step in the selection of a target domain within the target gene, the target gene, pre-mRNA, or mRNA sequence is examined for the presence of homopolymer runs (4 or more bases in a row) within the target sequence, the targeting of which should generally be avoided.

After selection of a target gene, marking the homopolymer runs, and identification of a target domain, a region of the pre-mRNA, mRNA or DNA sequence within that domain is analyzed. An analysis of the segment of the sequence extending approximately 5 to 100 bases or more upstream and downstream from a possible antisense target site is performed. The locations of potentially stable secondary structures are defined as stem-loop structures with predicted melting temperatures above 20° C. This analysis can be performed using commercially available software such as OLIGO 4.0 for Mac, or OligoTech. Sequences involved in stem hybridization for loop-stem secondary structure formation are regarded as part of the secondary structure and are treated as a structural unit for the purposes of analysis and selection of an antisense oligonucleotide. In one embodiment of this target selection technique, antisense oligonucleotides can be targeted to sequences of the gene, pre-mRNA, or mRNA with predicted secondary structure melting temperatures of less than 100° C. (according to the commercially available analysis programs). In a preferred embodiment, the secondary structure melting temperatures would be less than 60° C. In a further preferred embodiment, the secondary structure melting temperature would be less than 40° C. In a most preferred embodiment, secondary structure melting temperatures would be less than 20° C.

Oligonucleotide sequences with stable loop structures or which form stable dimers should be avoided. In one embodiment of this target selection technique, antisense oligonucleotides with predicted secondary structure melting temperatures of less than 100° C. (according to the commercially available analysis programs) are chosen. In a preferred embodiment the secondary structure melting temperatures would be less than 80° C. In a more preferred embodiment the secondary structure melting temperatures would be less than 60° C. In a further preferred embodiment the secondary structure melting temperatures would be less than 40° C. In the most preferred embodiment, the secondary structure melting temperature is less than 20° C. The formation of stable secondary or tertiary structure by the antisense oligonucleotides may potentially compete with their ability to bind to the target DNA or RNA. Similarly, formation of homodimers by the antisense oligonucleotides may compete with their ability to bind to target DNA or RNA. Homopolymer runs of a single base ideally will be three bases or less within the oligonucleotide sequence. Generally, within a target, those sequences with the lowest secondary structure melting temperatures (loop Tm), or no secondary structure work best.

Sequences of antisense oligonucleotides useful as compositions and in methods of the present invention include the following:

| | |
|---|---|
| Target Domain | |
| (Nucleotide Position #s) | Antisense Oligonucleotide Sequence |

Target Gene: PDE4A (PDE4A) - - Acc# U68532 (SEQ ID NO: 46)

| | | |
|---|---|---|
| 59-35 | tta gag cag gtc tcg cag aag aaa t, | (SEQ ID NO: 1) |
| 569-545 | agc gtc agc atg tat gtc acc atc g, | (SEQ ID NO: 2) |
| 886-862 | gct tgc tga ggt tct gga aga tgt c, | (SEQ ID NO: 3) |
| 1541-1518 | aga gct tcc tcg act cct gac aat, | (SEQ ID NO: 4) |
| 359-335 | atg tta gag ttg ttc agg ctg tta c, | (SEQ ID NO: 5) |
| 398-374 | agg agc tct tct tga tcg gtc ttc a, | (SEQ ID NO: 6) |
| 696-674 | gag aat ctc cag gtc cgt gaa ca, | (SEQ ID NO: 7) |
| 1350-1333 | ggc gct gta gta cca gtc, | (SEQ ID NO: 8) |
| 2797-2782 | aca ggg aca gag gtc t, | (SEQ ID NO: 9) |
| 3445-3421 | gac ttc tag tca gta tcg cca gga g, | (SEQ ID NO: 10) |

Target Gene: PDE4B (PDE4B2B) -- Acc# L20971 (SEQ ID NO: 47)

| | | |
|---|---|---|
| 720-696 | aca aat cac agt ggt gct ctg cct g, | (SEQ ID NO: 11) |
| 1441-1417 | ggt ctt cta aag tca tca tgt agg t, | (SEQ ID NO: 12) |
| 2185-2166 | cta agg tat cga gaa tgt cc, | (SEQ ID NO: 13) |
| 2506-2482 | cat gct cat caa gga tag aat gtt c, | (SEQ ID NO: 14) |
| 269-247 | gac gtt tgg gtt ata taa tac ac, | (SEQ ID NO: 15) |
| 995-971 | ttg tta gaa gcc atc tca ctg aca g, | (SEQ ID NO: 16) |
| 1787-1763 | atg tca ata acc atc ttc ctg agt g, | (SEQ ID NO: 17) |
| 1889-1865 | tct agg aga aga agc cct gaa ctt g, | (SEQ ID NO: 18) |
| 3254-3235 | gag ttc tat gca gac tct ca, | (SEQ ID NO: 19) |
| 3951-3927 | tca gta gtt cgg gag cat tca gaa g, | (SEQ ID NO: 20) |

Target Gene: PDE4C (PDE4C1B) -- Acc# Z46632 (SEQ ID NO: 48)

| | | |
|---|---|---|
| 119-102 | ttc tcc atg cgc cag aga, | (SEQ ID NO: 21) |
| 532-515 | cag agg agt tcc gag aca, | (SEQ ID NO: 22) |
| 1681-1664 | cga ggc ttg tca cct tct, | (SEQ ID NO: 23) |
| 2255-2231 | tgg ccc taa gtc ctc tgg ttg tcg a, | (SEQ ID NO: 24) |
| 69-52 | gct tgg ctg ctc cta ggc, | (SEQ ID NO: 25) |
| 560-538 | atg tcc tct cca tgt agg tcg ct, | (SEQ ID NO: 26) |
| 1140-1121 | gtt ccc act tac gtc cgc ca, | (SEQ ID NO: 27) |
| 1360-1336 | cca agt ctg tga aca cag cct cga g, | (SEQ ID NO: 28) |
| 2003-1979 | cga ttg tcc tcc agc gtg tcc agc a, | (SEQ ID NO: 29) |
| 3095-3071 | agt cat agc tct ctt cag cct cca a, | (SEQ ID NO: 30) |

Target Gene: PDE4D (PDE4D2A) -- Acc# U50158 (SEQ ID NO: 49)

| | | |
|---|---|---|
| 1683-1659 | ttg cac tgt tac gtg tca gga gaa c, | (SEQ ID NO: 31) |
| 1672-1659 | cgt gtc agg aga ac, | (SEQ ID NO: 32) |
| 1645-1621 | aca ggc ttc agg ttg gct ttc ctc t, | (SEQ ID NO: 33) |
| 1127-1103 | gag gct ttg ttg ggt tgc tca gat c, | (SEQ ID NO: 34) |
| 682-658 | aga taa tag cac atg agt aga ctg g, | (SEQ ID NO: 35) |
| 154-130 | cat ctc act gac gga gtg cct ggt c, | (SEQ ID NO: 36) |
| 609-592 | taa tgg tct tcg aga gtc, | (SEQ ID NO: 37) |
| 822-798 | ttg tac atc aag gca agt tca gag t, | (SEQ ID NO: 38) |
| 1046-1022 | gaa gaa ctc cag agc ttg tca ctt t, | (SEQ ID NO: 39) |
| 791-767 | tga tca gaa att gat tgg aca cac c, | (SEQ ID NO: 40) |
| 1371-1347 | tgg tac cat tca cga ttg tcc tcc a, | (SEQ ID NO: 41) |

Target Gene: PDE4D (PDE4D5A) -- Acc# AF012073 (SEQ ID NO: 50)

| | | |
|---|---|---|
| 300-276 | tct cgg aga gat cac tgg aga gag c, | (SEQ ID NO: 42) |
| 375-351 | atg tgc cac cgt gaa acg ccg ctg t, | (SEQ ID NO: 43) |

Target Gene: PDE4D (PDE4D3) -- Acc# L20970 (SEQ ID NO: 51)

| | | |
|---|---|---|
| 95-76 | tcc tac gtt aca tgt agt ga, | (SEQ ID NO: 44) |
| 178-159 | cat atc cag gaa tgc ctt ct, | (SEQ ID NO: 45) |

In Vivo Testing of Oligonucleotides

When used in the therapeutic treatment of disease, an appropriate dosage of an antisense PDE oligonucleotide, or mixture thereof, may be determined by any of several well established methodologies. For instance, animal studies are commonly used to determine the maximal tolerable dose, or MTD, of bio-active agent per kilogram weight. In general, at least one of the animal species tested is mammalian. Those skilled in the art regularly extrapolate doses for efficacy and avoiding toxicity to other species, including human. Additionally, therapeutic dosages may also be altered depending upon factors such as the severity of infection and the size or species of the host.

Oligonucleotides are preferably administered in a pharmaceutically acceptable carrier, via oral, intranasal, rectal, topical, intraperitoneal, intramuscular, subcutaneous, intracranial, subdermal, transdermal, intratracheal methods, or the like.

For example, topical diseases are preferably treated or prevented by formulations designed for topical application. Alternately, where the targeted disease state is in the stomach or gut, preparations of oligonucleotides may be provided by oral dosing. Additionally, pulmonary sites of disease, e.g., asthma, may be treated both parenterally and by direct application of suitably formulated forms of the oligonucleotides to the lung by inhalation therapy.

Where suitably formulated oligonucleotides are administered parenterally, the oligonucleotides can accumulate to relatively high levels in the kidneys, liver, spleen, lymph glands, adrenal gland, aorta, pancreas, bone marrow, heart, and salivary glands. Oligonucleotides also tend to accumulate to a lesser extent in skeletal muscle, bladder, stomach, esophagus, duodenum, fat, and trachea. Still lower concentrations are typically found in the cerebral cortex, brain stem, cerebellum, spinal cord, cartilage, skin, thyroid, and prostate (see generally Crooke, 1993, Antisense Research and Applications, CRC Press, Boca Raton, Fla.).

One of ordinary skill will appreciate that, from a medical practitioner's or patient's perspective, virtually any alleviation or prevention of an undesirable symptom would be desirable. Thus, the terms “treatment”, “therapeutic use”, or “medicinal use” used herein shall refer to any and all uses of the claimed oligonucleotides that remedy a disease state or symptoms, or otherwise prevent, hinder, retard, or reverse the progression of disease or other undesirable symptoms in any way whatsoever.

Preferably, animal hosts that may be treated using the oligonucleotides of the present invention include, but are not limited to, invertebrates, vertebrates, birds, mammals such as pigs, goats, sheep, cows, dogs, cats, and particularly humans. Oligonucleotides are designed to be appropriate to the particular animal to be treated.

Another embodiment of the presently described invention is the use of a therapeutic oligonucleotide that is additionally protonated/acidified so it can also serve as an antibiotic. Purified or crude nucleic acids can be protonated/acidified with acid, including but not limited to, phosphoric acid, nitric acid, hydrochloric acid, acetic acid, etc. For example, acid may be combined with nucleic acids in solution, or alternatively, the nucleic acids may be dissolved in an acidic solution. Excess acid may be removed by chromatography or in some cases by drying the nucleic acid.

Other procedures to prepare protonated nucleic acids known to the skilled artisan are equally contemplated to be within the scope of the invention. Once the nucleic acids of the present invention have been protonated they may be separated from any undesired components such as excess acid. The skilled artisan would know of many ways to separate the oligonucleotides from undesired components. For example, the oligonucleotide solution may be subjected to chromatography following protonation. In a preferred embodiment, the oligonucleotide solution is run over a poly(styrene-divinylbenzene) based resin column (e.g., Hamilton's PRP or Polymer Labs' PLRP) following protonation.

The protonated/acidified nucleic acids can be used directly, or in a preferred embodiment, processed further to remove any excess acid and salt via precipitation, reverse phase chromatography, diafiltration, or gel filtration. The protonated/acidified oligos can be concentrated by precipitation, lyophilization, solvent evaporation, etc. When suspended in water or saline, the acidified nucleic acid preparations of the invention typically exhibit a pH between 0.5 and 4.5 depending upon 1) the level of protonation/acidification, which can be determined by how much acid is used in the acidification process, and 2) the concentration of the nucleic acid. Alternatively, nucleic acids can be protonated by passage over a cation exchange column charged with hydrogen ions.

It has been discovered that protonated oligonucleotides, in a sequence independent manner, exhibit antibacterial properties. The protonated oligonucleotides of the invention are protonated/acidified to give a pH when dissolved in water of less than pH 7 to about pH 1, or in preferred embodiments, pH 6 to about 1 or pH 5 to about 1. In other preferred embodiments, the dissolved nucleic acids have a pH from pH 4.5 to about 1 or, in a preferred embodiment, a pH of 4.0 to about 1, or, in a more preferred embodiment, a pH of 3.0 to about 1, or, in another preferred embodiment, a pH of 2.0 to about 1.

Pharmaceutical Compositions and Delivery

Oligonucleotides may be formulated with a variety of physiological carrier molecules for in vivo use. For example, the oligonucleotides may be combined with a lipid, cationic lipid, or anionic lipid (which may be preferred for protonated/acidified oligonucleotides) and the resulting oligonucleotide/lipid emulsion, or liposomal suspension may, inter alia, effectively increase the in vivo half-life of the oligonucleotide.

When used in the therapeutic treatment of disease, an appropriate dosage of a PDE4 modulating oligonucleotide, or mixture thereof, may be determined by any of several well established methodologies. For instance, animal studies are commonly used to determine the maximal tolerable dose, or MTD, of bio-active agent per kilogram weight. In general, at least one of the animal species tested is mammalian. Those skilled in the art regularly extrapolate doses for efficacy and avoiding toxicity to other species, including human. Additionally, therapeutic dosages may also be altered depending upon factors such as the severity of the disease, and the size or species of the host.

The use of protonated/acidified oligonucleotides facilitates their encapsulation in anionic lipids which are thought to have a number of advantages over cationic liposomes (R. J. Lee and L. Huang, “Lipidic Vector Systems for Gene Transfer”, in Critical Reviews in Therapeutic Drug Carrier Systems 14(2):173-206 (1997)). Specifically, anionic liposomes are likely to be less toxic than cationic liposomes, they exhibit lower non-specific uptake, and they can be targeted with the appropriate ligands to specific cells.

Examples of suitable anionic lipids for use with protonated/acidified oligonucleotides include, but are not limited to, cardiolipin, dimyristoyl, dipalmitoyl, or dioleoyl phosphatidyl choline or phosphatidyl glycerol, palmitoyloleoyl phosphatidyl choline or phosphatidyl glycerol, phosphatidic acid, lysophosphatidic acid, phosphatidyl serine, phosphatidyl inositol, and anionic forms of cholesterol. The use of cationic, anionic, and/or neutral lipid compositions or liposomes is generally described in International Publications Nos. WO 90/14074, WO 91/16024, WO 91/17424, and U.S. Pat. No. 4,897,355, herein incorporated by reference.

By assembling oligonucleotides into lipid-associated structures, the oligonucleotides may be targeted to specific cell types by the incorporation of suitable targeting agents (i.e., specific antibodies or receptors) into the oligonucleotide/lipid complex.

Pharmaceutical compositions containing oligonucleotides of the invention in intimate admixture with a pharmaceutical carrier can be prepared according to conventional pharmaceutical compounding techniques. The carrier may take a wide variety of forms depending on the form of preparation desired for administration, e.g., intravenous, oral, topical, aerosol (for topical or inhalation therapy), suppository, parenteral, or spinal injection.

In preparing the compositions in oral dosage form, any of the usual pharmaceutical media may be employed, such as, for example, water, glycols, oils, alcohols, flavoring agents, preservatives, coloring agents and the like in the case of oral liquid preparations (such as, for example, suspension, elixirs, and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques.

For parenteral application by injection, preparations may comprise an aqueous solution of a water soluble, or solubilized, and pharmaceutically acceptable form of the oligonucleotide in an appropriate saline solution. Injectable suspensions may also be prepared using appropriate liquid carriers, suspending agents, agents for adjusting the isotonicity, preserving agents, and the like. Actual methods for preparing parenterally administrable compositions and adjustments necessary for administration to subjects will be shown or apparent to those skilled in the art and are described in more detail in, for example, Remington's Pharmaceutical Science, 15th Ed., Mack Publishing Company, Easton, Pa. (1980), which is incorporated herein by reference. The presently described oligonucleotides should be parenterally administered at concentrations below the maximal tolerable dose (MTD) established for the oligonucleotides.

For topical administration, the carrier may take a wide variety of forms depending on the preparation, which may be a cream, dressing, gel, lotion, ointment, or liquid.

Aerosols are prepared by dissolving or suspending the oligonucleotide in a propellant such as ethyl alcohol or in propellant and solvent phases. The pharmaceutical compositions for topical or aerosol form will generally contain from about 0.01% by weight (of the oligonucleotide) to about 40% by weight, preferably about (0.02% to about 10% by weight, and more preferably about 0.05% to about 5% by weight depending on the particular form employed.

Suppositories are prepared by mixing the oligonucleotide with a lipid vehicle such as theobroma oil, cacao butter, glycerin, gelatin, or polyoxyethylene glycols.

The presently described oligonucleotides may be administered to the body by virtually any means used to administer conventional therapeutics. A variety of delivery systems are well known in the art for delivering bioactive compounds to an animal. These systems include, but are not limited to, intravenous or intramuscular or intra-tracheal injection, nasal spray, aerosols for inhalation, and oral or suppository administration. The specific delivery system used depends on the location of the disease, and it is well within the skill of one in the art to determine the location of the disease and to select an appropriate delivery system.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g., amounts, temperature, concentrations, etc.) but some experimental errors and deviations should be allowed for. Unless otherwise indicated, parts are parts by weight, molecular weight is average molecular weight, temperature is in degrees centigrade, and pressure is at or near ambient.

### Example 1

### B Cell Mediated Dermatitis

An anti-inflammatory oligonucleotide, OE-2a (SEQ ID NO.: 32), is a 2′ O-methyl RNA phosphodiester linked, with 5′ and 3′ ends blocked with inverted Ts, and is targeted against the human PDE4 gene. OE-2a was dissolved at 3 5 mg/ml (7.7 μMolar) in water at approximately pH 3 and used to treat a 37 year old male with a history of severe recurrent atopic dermatitis. The dermatitis was specifically eradicated with OE-2a. In addition, fiture occurrences of atopic dermatitis have been completely eliminated.

A 37 year old male presented with severe atopic dermatitis, completely covering the inside of both forearms. Atopic dermatitis is a chronic disorder characterized by intensely pruritic inflamed papules. The inflammatory response is associated with over-production of IgE by B lymphocytes. Higher levels of PDE4 have been reported for individuals with atopic dermatitis. An antisense oligonucleotide, OE-2a, specifically targeted to PDE4 was applied to a 2″ by 3″ segment of the left forearm. A second oligo, OE-1, was applied to another segment of the same arm. OE-1 has a similar base distribution to OE-2a, but is homologous to a bacterial gene. Oligonucleotides were applied twice at 12 hour intervals.

OE-2a was successful at clearing the dermatitis on the area to which it was applied. OE-1 had no effect. The patient commented prior to the second treatment that the itching had stopped on the area corresponding to the OE-2a after about 6 hours.

Treatment on the second arm was initiated at this time exclusively with OE-2a. Again there were 2 treatments 12 hours apart. The patient remarked that all itching had ceased within 6 hours. The dermatitis was completely cleared within 12 hours of the second treatment.

There have been no recurrences of atopic dermatitis on this individual from February through December of a single year, although he typically suffers from recurring bouts during the heat of the summer.

### Example 2

### T Cell Mediated Dermatitis

OE-2a at 35 mg/ml (7.7 μMolar) in water at approximately pH 3 was used to treat a case of T cell mediated contact dermatitis triggered by poison ivy. The dermatitis was completely eliminated with 2 treatments, and secondary eruptions on other areas of the infected individual were prevented.

A female presented with poison ivy on several areas of the bottom of her foot. The patient complained of itching that is associated with T cell mediated type hypersensitivity. The contact dermatitis was characterized by redness, induration and vesiculation. The anti-inflammatory oligo OE-2a was applied to the bottom of the foot. Within 12 hours the patient noted that the intense itching had ceased. A second application of OE-2a resulted in a disappearance of the redness, induration, and vesiculation. It is of interest to note that topical application of OE-2a not only eliminated the visible poison ivy on the bottom of the foot, but also prevented any additional eruptions of poison ivy induced dermatitis elsewhere on the individual.

### Example 3

### Acute Wheal and Flare Reaction

OE-2a at 43.6 mg/ml (7.7 μMolar) and approximately pH 7 was able to prevent both an immediate and delayed-type hypersensitivity response when applied within minutes of receiving multiple wasp stings.

A female patient with a history of severe wheal and flare skin reaction in response to insect bites presented with an arm that had received multiple wasp stings. The patient was in severe pain and hysterical. OE-2a was administered immediately. Within 5 minutes the patient was calm and pain free. Remarkably, administration of the OE-2a within 5 minutes of receiving the stings completely prevented any immediate wheat and flare reaction, as well as any delayed reaction.

### Example 4

### Chemically Induced Dermatitis

OE-2a was successfully used to treat a case of chemically induced contact dermatitis that had failed to respond to standard dermatological treatments.

A 59 year old woman presented with a case of contact dermatitis on her neck triggered by a reaction to dry cleaning reagents in a turtle neck sweater. Over the course of a month she had been treated by a dermatologist with cortisone, antibiotics, and a variety of other topical drugs. The various treatments gave no more than temporary sporadic relief and they failed to eliminate the itching and redness. At this time the patient was treated topically with OE-2a. The itching and redness disappeared completely after two treatments with no recurrences over a 5 month period.

### Example 5

### Intranasal Administration of PDE-4

Several healthy male volunteers with severe allergic rhinitis were treated with OE-2a. The males ranged in age from 45 years old to 73 years old. The weights ranged from 175 pounds to 230 pounds. A sterile nasal spray container was filled with a solution containing OE-2a dissolved in normal saline. The solution of oligonucleotide in saline, at a concentration of concentration of approximately 300 A₂60 per ml, was administered via the intranasal route twice daily to each nostril. This dosage regime was continued for three days.

Upon examination, following the three day course of treatment, all volunteers were found to be completely free of all symptoms of allergic rhinitis. Specifically there was no nasal congestion, no nasal discharge, no sneezing, no difficulty breathing through the nose, and the nasal passages appeared to be completely clear. There has been no relapse of any of the symptoms of rhinitis since treatment schedule was completed, although the volunteers have continued to be exposed to the allergens that triggered the rhinitis. In conclusion, intranasal administration of PDE-4 dissolved in saline and administered via an intranasal spray appeared to cure allergic rhinitis in healthy male volunteers after only three days of treatment, with no further recurrences of the symptoms.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An acid resistant oligonucleotide targeted to an RNA encoding a phosphodiesterase 4 (PDE4) protein selected from the group consisting of SEQ ID NO:46, SEQ ID NO:47, SEQ ID NO:48, SEQ ID NO:49, SEQ ID NO:50 and SEQ ID NO:51, said oligonucleotide comprising:
a polymer of nucleotides, said polymer having a nucleic acid backbone structure modified from that of a naturally occurring nucleotide polymer; and
a blocking chemical modification at or near at least the 3′ end of the polymer,
wherein the oligonucleotide is characterized by a pH stability of at least one hour at a pH of about 0.01 to about 10 and a nuclease resistance of at least twice that of a naturally occurring polymer having the same number of nucleotides.

2. The oligonucleotide of claim 1, wherein the oligonucleotide has from about one to about 100 nucleotides.

3. The oligonucleotide of claim 1, wherein the oligonucleotide is completely or partially derivatized by a chemical moiety selected from the group consisting of: phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate linkages, sulfone intemucleotide linkages, 3′-3′ linkages, 5′-2′ linkages, 5′-5′ linkages, 2′-deoxy-erythropentofuranosyl, 2′-fluoro, 2′-O-alkyl nucleotides, 2′-O-alkyl-n(O-alkyl) phosphodiesters, 2′-O-methyl nucleotides, morpholino linkages, p-ethoxy oligonucleotides, PNA linkages, p-isopropyl oligonucleotides, butanol, butyl, and phosphoramidates

4. The oligonucleotide of claim 1, wherein the oligonucleotide has a sequence selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; and SEQ ID NO: 45.

5. The oligonucleotide of claim 1, wherein the oligonucleotide binds selectively to exon/intron boundaries and splice sites of RNA.

6. The oligonucleotide of claim 1, wherein the oligonucleotide binds selectively to an mRNA encoding a phosphodiesterase E4 protein.

7. An acid resistant oligonucleotide that binds selectively to DNA involved in phosphodiesterase 4 (PDE4) expression, said oligonucleotide comprising:
a polymer of nucleotides, said polymer having a nucleic acid backbone structure modified from that of a naturally occurring nucleotide polymer; and
a blocking chemical modification at or near at least the 3′ end of the polymer;
wherein the oligonucleotide is characterized by a pH stability of at least one hour at a pH of about 0.1 to about 10 and a nuclease resistance of at least twice that of a naturally occurring polymer having the same number of nucleotides.

8. The oligonucleotide of claim 7, wherein the DNA is selected from the group consisting of: PDE4 coding sequences, PDE4 promoter sequences, and PDE4 enhancer sequences.

9. The oligonucleotide of claim 7, wherein the oligonucleotide has from about one to about 100 nucleotides.

10. The oligonucleotide of claim 7, wherein the oligonucleotide is completely or partially derivatized by a chemical moiety selected from the group consisting of: phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate linkages, sulfone internucleotide linkages, 3′-3′ linkages, 5′-2′ linkages, 5′-5′ linkages, 2′-deoxy-erythropentofuranosyl, 2′-fluoro, 2′-O-alkyl nucleotides, 2′-O-alkyl-n(O-alkyl) phosphodiesters, 2′-O-methyl nucleotides, morpholino linkages, p-ethoxy oligonucleotides, PNA linkages, p-isopropyl oligonucleotides, butanol, butyl, and phosphoramidates.

11. A pharmaceutical composition comprised of an acid resistant oligonucleotide that binds selectively to an mRNA encoding a phosphodiesterase 4 protein, said oligonucleotide comprising a polymer having a nucleic acid backbone structure modified from that of a naturally occurring nucleotide polymer and a blocking chemical modification at or near at least one end of the polymer; and
a pharmaceutically acceptable carrier;
wherein the oligonucleotide is characterized by a pH stability of at least one hour at a pH of about 0.01 to about 10 and a nuclease resistance of at least twice that of a naturally occurring polymer having the same number of nucleotides.

12. The pharmaceutical composition of claim 11, wherein the nucleic acid is an oligonucleotide having from about one to about 100 nucleotides.

13. The pharmaceutical composition of claim 12, wherein said nucleic acid is linked to a compound selected from the group consisting of protein, amino acid, lipid, sugar, glycoprotein, antibiotic, organic compound, organometallic compound, steroid, and metal.

14. The pharmaceutical composition of claim 11, wherein said nucleic acid is protonated.

15. The pharmaceutical composition of claim 11, wherein the oligonucleotide has been protonated/acidified to have a pH at or below 7.

16. The pharmaceutical composition of claim 11, wherein the nucleic acid is encapsulated in a liposome.

17. A pharmaceutical composition comprised of an acid resistant oligonucleotide that binds selectively to DNA involved in phosphodiesterase 4 expression, said oligonucleotide comprising a polymer having a nucleic acid backbone structure modified from that of a naturally occurring nucleotide polymer and a blocking chemical modification at or near at least one end of the polymer; and
a pharmaceutically acceptable carrier;
wherein the oligonucleotide is characterized by a pH stability of at least one hour at a pH of about 0. 1 to about 10 and a nuclease resistance of at least twice that of a naturally occurring polymer having the same number of nucleotides.

18. The pharmaceutical composition of claim 17, wherein the nucleic acid is an oligonucleotide having from about one to about 100 nucleotides.

19. The pharmaceutical composition of claim 17, wherein said nucleic acid is linked to a compound selected from the group consisting of protein, amino acid, lipid, sugar, glycoprotein, antibiotic, organic compound, organometallic compound, steroid, and metal.

20. The pharmaceutical composition of claim 17, wherein said nucleic acid is protonated.

21. The pharmaceutical composition of claim 20, wherein the oligonucleotide has been protonated/acidified to have a pH at or below 7.

22. The pharmaceutical composition of claim 17, wherein the nucleic acid is encapsulated in a liposome.

23. A method of treating a mammal comprising topically administering to a site of need a therapeutically effective amount of an acid resistant oligonucleotide that binds selectively to an mRNA encoding a phosphodiesterase 4 protein, said oligonucleotide comprising a polymer having a nucleic acid backbone structure modified from that of a naturally occurring nucleotide polymer and a blocking chemical modification at or near at least the 3′ end of the polymer;
wherein the oligonucleotide is characterized by a pH stability of at least one hour at a pH of about 0.01 to about 10 and a nuclease resistance of at least twice that of a naturally occurring polymer having the same number of nucleotides.

24. The method of claim 11, wherein the polymer is completely or partially derivatized by a chemical moiety selected from the group consisting of: phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate and/or sulfone internucleotide linkages, 3′-3′ linkages, 2′-5′ linkages, 5′-5′ linkages, 2′-deoxy-erythropentofuranosyl, 2′-fluoro, 2′-O-alkyl nucleotides, 2′-O-alkyl-n(O-alkyl) phosphodiesters, 2′-O-methyl nucleotides, morpholino linkages, p-ethoxy oligonucleotides, PNA linkages, p-isopropyl oligonucleotides, butanol, butyl, and phosphoramidates.

25. The method of claim 23, wherein the oligonucleotide is protonated.

26. The method of claim 23, wherein the PDE4 is selected from the group consisting of SEQ ID NO:46, SEQ ID NO:47, SEQ ID NO:48, SEQ ID NO:49, SEQ ID NO:50 and SEQ ID NO:51.

27. The method of claim 23 wherein the oligonucleotide is administered to a mammal suffering from a disease or disorder selected from the group consisting of: atopic dermatitis, allergic rhino-conjunctivitis, T cell mediated dermatitis, B-cell mediated dermatitis, and acute wheal and flare reaction.

28. A method of treating a mammal comprising topically administering to a site of need a therapeutically effective amount of an acid resistant oligonucleotide that binds selectively to DNA involved in phosphodiesterase 4 expression, said oligonucleotide comprising a polymer having a nucleic acid backbone structure modified from that of a naturally occurring nucleotide polymer and a blocking chemical modification at or near at least the 3′ end of the polymer;
wherein the oligonucleotide is characterized by a pH stability of at least one hour at a pH of about 0.1 to about 10 and a nuclease resistance of at least twice that of a naturally occurring polymer having the same number of nucleotides.

29. The method of claim 28, wherein the DNA involved in phosphodiesterase 4 expression is selected from the group consisting of: PDE4 coding sequences, PDE4 promoter sequences, and PDE4 enhancer sequences.

30. The method of claim 28, wherein the polymer is completely or partially derivatized by a chemical moiety selected from the group consisting of: phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate and/or sulfone internucleotide linkages, 3′-3′ linkages, 2′-5′ linkages, 5′-5′ linkages, 2′-deoxy-erythropentofuranosyl, 2′-fluoro, 2′-O-alkyl nucleotides, 2′-O-methyl nucleotides, 2′-O-alkyl-n(O-alkyl) phosphodiesters, morpholino linkages, p-ethoxy oligonucleotides, PNA linkages, p-isopropyl oligonucleotides, butanol, butyl, and phosphoramidates and phosphoramidates.

31. The method of claim 28, wherein the oligonucleotide is protonated.

32. A method of treating a mammal comprising intranasally administering a therapeutically effective amount of an acid resistant oligonucleotide that binds selectively to an mRNA encoding a phosphodiesterase 4 protein, said oligonucleotide comprising a polymer having a nucleic acid backbone structure modified from that of a naturally occurring nucleotide polymer and a blocking chemical modification at or near at least the 3′ end of the polymer;
wherein the oligonucleotide is characterized by a pH stability of at least one hour at a pH of about 0.01 to about 10 and a nuclease resistance of at least twice that of a naturally occurring polymer having the same number of nucleotides.

33. The method of claim 32, wherein the polymer is completely or partially derivatized by a chemical moiety selected from the group consisting of: phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate and/or sulfone intemucleotide linkages, 3′-3′ linkages, 2′-5′ linkages, 5′-5′ linkages, 2′-deoxy-erythropentofuranosyl, 2′-fluoro, 2′-O-alkyl nucleotides, 2′-O-alkyl-n(O-alkyl) phosphodiesters, 2′-O-methyl nucleotides, morpholino linkages, p-ethoxy oligonucleotides, PNA linkages, p-isopropyl oligonucleotides, butanol, butyl, and phosphoramidates.

34. The method of claim 32, wherein the oligonucleotide is protonated.

35. The method of claim 32, wherein the PDE4 is selected from the group consisting of: SEQ ID NO:46, SEQ ID NO:47, SEQ ID NO:48, SEQ ID NO:49, SEQ ID NO:50 and SEQ ID NO:51.

36. The method of claim 35, wherein the oligonucleotide has a sequence selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; and SEQ ID NO: 45.

37. The method of claim 35, wherein the oligonucleotide is administered to a mammal suffering from a disease or disorder selected from the group consisting of: atopic dermatitis, allergic rhino-conjunctivitis, T cell mediated dermatitis, B-cell mediated dermatitis, and acute wheal and flare reaction.

38. A method of treating a mammal comprising intranasally administering a therapeutically effective amount of an acid resistant oligonucleotide that binds selectively to DNA involved in phosphodiesterase 4 expression, said oligonucleotide comprising a polymer having a nucleic acid backbone structure modified from that of a naturally occurring nucleotide polymer and a blocking chemical modification at or near at least the 3′ end of the polymer;
wherein the oligonucleotide is characterized by a pH stability of at least one hour at a pH of about 0.1 to about 10 and a nuclease resistance of at least twice that of a naturally occurring-polymer having the same number of nucleotides.

39. The method of claim 38, wherein the DNA involved in phosphodiesterase 4 expression is selected from the group consisting of: PDE4 coding sequences, PDE4 promoter sequences, and PDE4 enhancer sequences.

40. The method of claim 38, wherein the polymer is completely or partially derivatized by a chemical moiety selected from the group consisting of: phosphodiester linkages, phosphotriester linkages, phosphoramidate linkages, siloxane linkages, carbonate linkages, carboxymethylester linkages, acetamidate linkages, carbamate linkages, thioether linkages, bridged phosphoramidate linkages, bridged methylene phosphonate linkages, phosphorothioate linkages, methylphosphonate linkages, phosphorodithioate linkages, morpholino, bridged phosphorothioate and/or sulfone intemucleotide linkages, 3′-3′ linkages, 2′-5′ linkages, 5′-5′ linkages, 2′-deoxy-erythropentofuranosyl, 2′-fluoro, 2′-O-alkyl nucleotides, 2′-O-methyl nucleotides, 2′-O-alkyl-n(O-alkyl) phosphodiesters, morpholino linkages, p-ethoxy oligonucleotides, PNA linkages, p-isopropyl oligonucleotides, butanol, butyl, and phosphoramidates and phosphoramidates.

41. The method of claim 38, wherein the oligonucleotide is protonated.


SEQUENCE LISTING (51 sequences)

SEQ ID NO: 1 - DNA, 25 bp, Artificial Sequence
  Synthesized Oligonucleotide
ttagagcaggtctcgcagaagaaat

SEQ ID NO: 2 - DNA, 25 bp, Artificial Sequence
  Synthesized Oligonucleotide
agcgtcagcatgtatgtcaccatcg

SEQ ID NO: 3 - DNA, 25 bp, Artificial Sequence
  Synthesized Oligonucleotide
gcttgctgaggttctggaagatgtc

SEQ ID NO: 4 - DNA, 24 bp, Artificial Sequence
  Synthesized Oligonucleotide
agagcttcctcgactcctgacaat

SEQ ID NO: 5 - DNA, 25 bp, Artificial Sequence
  Synthesized Oligonucleotide
atgttagagttgttcaggctgttac

SEQ ID NO: 6 - DNA, 25 bp, Artificial Sequence
  Synthesized Oligonucleotide
aggagctcttcttgatcggtcttca

SEQ ID NO: 7 - DNA, 23 bp, Artificial Sequence
  Synthesized Oligonucleotide
gagaatctccaggtccgtgaaca

SEQ ID NO: 8 - DNA, 18 bp, Artificial Sequence
  Synthesized Oligonucleotide
ggcgctgtagtaccagtc

SEQ ID NO: 9 - DNA, 16 bp, Artificial Sequence
  Synthesized Oligonucleotide
acagggacagaggtct

SEQ ID NO: 10 - DNA, 25 bp, Artificial Sequence
  Synthesized Oligonucleotide
gacttctagtcagtatcgccaggag

SEQ ID NO: 11 - DNA, 25 bp, Artificial Sequence
  Synthesized Oligonucleotide
acaaatcacagtggtgctctgcctg

SEQ ID NO: 12 - DNA, 25 bp, Artificial Sequence
  Synthesized Oligonucleotide
ggtcttctaaagtcatcatgtaggt

SEQ ID NO: 13 - DNA, 20 bp, Artificial Sequence
  Synthesized Oligonucleotide
ctaaggtatcgagaatgtcc

SEQ ID NO: 14 - DNA, 25 bp, Artificial Sequence
  Synthesized Oligonucleotide
catgctcatcaaggatagaatgttc

SEQ ID NO: 15 - DNA, 23 bp, Artificial Sequence
  Synthesized Oligonucleotide
gacgtttgggttatataatacac

SEQ ID NO: 16 - DNA, 25 bp, Artificial Sequence
  Synthesized Oligonucleotide
ttgttagaagccatctcactgacag

SEQ ID NO: 17 - DNA, 25 bp, Artificial Sequence
  Synthesized Oligonucleotide
atgtcaataaccatcttcctgagtg

SEQ ID NO: 18 - DNA, 25 bp, Artificial Sequence
  Synthesized Oligonucleotide
tctaggagaagaagccctgaacttg

SEQ ID NO: 19 - DNA, 20 bp, Artificial Sequence
  Synthesized Oligonucleotide
gagttctatgcagactctca

SEQ ID NO: 20 - DNA, 25 bp, Artificial Sequence
  Synthesized Oligonucleotide
tcagtagttcgggagcattcagaag

SEQ ID NO: 21 - DNA, 18 bp, Artificial Sequence
  Synthesized Oligonucleotide
ttctccatgcgccagaga

SEQ ID NO: 22 - DNA, 18 bp, Artificial Sequence
  Synthesized Oligonucleotide
cagaggagttccgagaca

SEQ ID NO: 23 - DNA, 18 bp, Artificial Sequence
  Synthesized Oligonucleotide
cgaggcttgtcaccttct

SEQ ID NO: 24 - DNA, 25 bp, Artificial Sequence
  Synthesized Oligonucleotide
tggccctaagtcctctggttgtcga

SEQ ID NO: 25 - DNA, 18 bp, Artificial Sequence
  Synthesized Oligonucleotide
gcttggctgctcctaggc

SEQ ID NO: 26 - DNA, 23 bp, Artificial Sequence
  Synthesized Oligonucleotide
atgtcctctccatgtaggtcgct

SEQ ID NO: 27 - DNA, 20 bp, Artificial Sequence
  Synthesized Oligonucleotide
gttcccacttacgtccgcca

SEQ ID NO: 28 - DNA, 25 bp, Artificial Sequence
  Synthesized Oligonucleotide
ccaagtctgtgaacacagcctcgag

SEQ ID NO: 29 - DNA, 25 bp, Artificial Sequence
  Synthesized Oligonucleotide
cgattgtcctccagcgtgtccagca

SEQ ID NO: 30 - DNA, 25 bp, Artificial Sequence
  Synthesized Oligonucleotide
agtcatagctctcttcagcctccaa

SEQ ID NO: 31 - DNA, 25 bp, Artificial Sequence
  Synthesized Oligonucleotide
ttgcactgttacgtgtcaggagaac

SEQ ID NO: 32 - DNA, 14 bp, Artificial Sequence
  Synthesized Oligonucleotide
cgtgtcaggagaac

SEQ ID NO: 33 - DNA, 25 bp, Artificial Sequence
  Synthesized Oligonucleotide
acaggcttcaggttggctttcctct

SEQ ID NO: 34 - DNA, 25 bp, Artificial Sequence
  Synthesized Oligonucleotide
gaggctttgttgggttgctcagatc

SEQ ID NO: 35 - DNA, 25 bp, Artificial Sequence
  Synthesized Oligonucleotide
agataatagcacatgagtagactgg

SEQ ID NO: 36 - DNA, 25 bp, Artificial Sequence
  Synthesized Oligonucleotide
catctcactgacggagtgcctggtc

SEQ ID NO: 37 - DNA, 18 bp, Artificial Sequence
  Synthesized Oligonucleotide
taatggtcttcgagagtc

SEQ ID NO: 38 - DNA, 25 bp, Artificial Sequence
  Synthesized Oligonucleotide
ttgtacatcaaggcaagttcagagt

SEQ ID NO: 39 - DNA, 25 bp, Artificial Sequence
  Synthesized Oligonucleotide
gaagaactccagagcttgtcacttt

SEQ ID NO: 40 - DNA, 25 bp, Artificial Sequence
  Synthesized Oligonucleotide
tgatcagaaattgattggacacacc

SEQ ID NO: 41 - DNA, 25 bp, Artificial Sequence
  Synthesized Oligonucleotide
tggtaccattcacgattgtcctcca

SEQ ID NO: 42 - DNA, 25 bp, Artificial Sequence
  Synthesized Oligonucleotide
tctcggagagatcactggagagagc

SEQ ID NO: 43 - DNA, 25 bp, Artificial Sequence
  Synthesized Oligonucleotide
atgtgccaccgtgaaacgccgctgt

SEQ ID NO: 44 - DNA, 18 bp, Artificial Sequence
  Synthesized Oligonucleotide
tcctacgttacatgtagt

SEQ ID NO: 45 - DNA, 20 bp, Artificial Sequence
  Synthesized Oligonucleotide
catatccaggaatgccttct

SEQ ID NO: 46 - protein, 647 aa, human
MPLVDFFCETCSKPWLVGWWDQFKRMLNRELTHLSEMSRSGNQVSEYISTTFLDKQNEVEIPSPTMKEREKQQAPRPRPSQPPPPPVPHLQPMSQITGLKKLMHSNSLNNSNIPRFGVKTDQEELLAQELENLNKWGLNIFCVSDYAGGRSLTCIMYMIFQERDLLKKFRIPVDTMVTYMLTLEDHYHADVAYHNSLHAADVLQSTHVLLATPALDAVFTDLEILAALFAAAIHDVDHPGVSNQFLINTNSELALMYNDESVLENHHLAVGFKLLQEDNCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQEILDTLEDNRDWYYSAIRQSPSPPPEEESRGPGHPPLPDKFQFELTLEEEEEEEISMAQIPCTAQEALTAQGLSGVEEALDATIAWEASPAQESLEVMAQEASLEAELEAVYLTQQAQSTGSAPVAPDEFSSREEFVVAVSHSSPSALALQSPLLPAWRTLSVSEHAPGLPGLPSTAAEVEAQREHQAAKRACSACAGTFGEDTSALPAPGGGGSGGDPT

SEQ ID NO: 47 - protein, 712 aa, human
MENLGVGEGAEACSRLSRSRGRHSMTRAPKHLWRQPRRPIRIQQRFYSDPDKSAGCRERDLSPRPELRKSRLSWPVSSCRRFDLENGLSCGRRALDPQSSPGLGRIMQAPVPHSQRRESFLYRSDSDYELSPKAMSRNSSVASDLHGEDMIVTPFAQVLASLRTVRSNVAALARQQCLGAAKQGPVGNPSSSNQLPPAEDTGQKLALETLDELDWCLDQLETLQTRHSVGEMASNKFKRILNRELTHLSETSRSGNQVSEYISRTFLDQQTEVELPKVTAEEAPQPMSRISGLHGLCHSASLSSATVPRFGVQTDQEEQLAKELEDTNKWGLDVFKVADVSGNRPLTAIIFSIFQERDLLKTFQIPADTLATYLLMLEGHYHANVAYHNSLHAADVAQSTHVLLATPALEAVFTDLEILAALFASAIHDVDHPGVSNQFLINTNSDVALMYNDASVLENHHLAVGFKLLQAENCDIFQNLSAKQRLSLRRMVIDMVLATDMSKHMNLLADLKTMVETKKVTSLGVLLLDNYSDRIQVLQNLVHCADLSNPTKPLPLYRQWTDRIMAEFFQQGDRERESGLDISPMCDKHTASVEKSQVGFIDYIAHPLWETWADLVHPDAQDLLDTLEDNREWYQSKIPRSPSDLTNPERDGPDRFQFELTLEEAEEEDEEEEEEGEETALAKEALELPDTELLSPEAGPDPGDLPLDNQRT

SEQ ID NO: 48 - protein, 564 aa, human
MKEHGGTFSSTGISGGSGDSAMDSLQPLQPNYMPVCLFAEESYQKLAMETLEELDWCLDQLETIQTYRSVSEMASNKFKRMLNRELTHLSEMSRSGNQVSEYISNTFLDKQNDVEIPSPTQKDREKKKKQQLMTQISGVKKLMHSSSLNNTSISRFGVNTENEDHLAKELEDLNKWGLNIFNVAGYSHNRPLTCIMYAIFQERDLLKTFRISSDTFITYMMTLEDHYHSDVAYHNSLHAADVAQSTHVLLSTPALDAVFTDLEILAAIFAAAIHDVDHPGVSNQFLINTNSELALMYNDESVLENHHLAVGFKLLQEEHCDIFMNLTKKQRQTLRKMVIDMVLATDMSKHMSLLADLKTMVETKKVTSSGVLLLDNYTDRIQVLRNMVHCADLSNPTKSLELYRQWTDRIMEEFFQQGDKERERGMEISPMCDKHTASVEKSQVGFIDYIVHPLWETWADLVQPDAQDILDTLEDNRNWYQSMIPQSPSPPLDEQNRDCQGLMEKFQFELTLDEEDSEGPEKEGEGHSYFSSTKTLCVIDPENRDSLGETDIDIATEDKSPVDT

SEQ ID NO: 49 - protein, 507 aa, human [sequence omitted]

SEQ ID NO: 50 - protein, 745 aa, human
MAQQTSPDTLTVPEVDNPHCPNPWLNEDLVKSLRENLLQHEKSKTARKSVSPKLSPVISPRNSPRLLRRMLLSSNIPKQRRFTVAHTCFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 51 - protein, 673 aa, human
MMHVNNFPFRRHSWICFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT